(19) United States
(12) Patent Application Publication
(10) Pub. No.: US 2008/0269248 A1
(43) Pub. Date: Oct. 30, 2008

(54) Tartrate and Malate Salts of Trans-1-((1R,3S)-6-Chloro-3-Phenylindan-1-Yl)-3,3-Dimethylpiperazine

(75) Inventors: Ole Nielsen, Valby (DK); Heidi Lopez de Diego, Naerum (DK); Benny Bang-Andersen, Copenhagen S. (DK)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. Lundbeck A/S, Valby-Copenhagen (DK)

(21) Appl. No.: 11/816,403

(22) PCT Filed: Feb. 14, 2006
(86) PCT No.: PCT/DK2006/000087

§ 371 Date: Mar. 12, 2008

Related U.S. Application Data
(60) Provisional application No. 60/653,418, filed on Feb. 16, 2005.

(30) Foreign Application Priority Data
Feb. 16, 2005 (DK) PA200500238

(51) Int. Cl. A61K 31/495; C07D 241/04; A61P 25/00

(52) U.S. Cl. 514/255.03; 544/403

(57) ABSTRACT

A tartrate and malate salt of trans-1-(6-chloro-3-phenylindan-1-yl)-3,3-dimethylpiperazine, in particular for medical use, pharmaceutical formulations thereof, including for treatment of schizophrenia or other diseases involving psychotic symptoms.

The present invention relates to a tartrate and malate salt of trans-1-(6-chloro-3-phenylindan-1-yl)-3,3-dimethylpiperazine, in particular for medical use, pharmaceutical formulations of these salts, including for treatment of schizophrenia or other diseases involving psychotic symptoms.

### BACKGROUND OF THE INVENTION

The compound, which is the subject of the present invention (Compound I, trans-1-((1R,3S)-6-chloro-3-phenylindan-1-yl)-3,3-dimethylpiperazine) has the general formula (I).

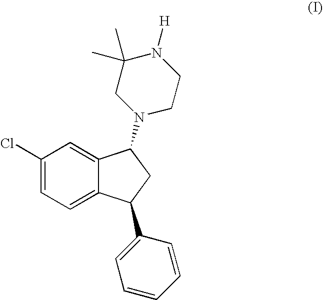

Compound I and salts thereof, including a fumarate and maleate salt, are described in PCT/DK04/000546 (WO05/016901).

As described in PCT/DK04/000546 the inventors have found that Compound I displays high affinity for dopamine (DA) D1 receptors, DA D2 receptors and for alfal adrenoceptors. Furthermore, Compound I was found to be an antagonist at dopamine D1 and D2 receptors, and at serotonin 5-HT2a receptors. As further described in PCT/DK04/000546, Compound I is a relatively weak inhibitor of CYP2D6 (i.e. reduced potential for drug to drug interaction) and has a relatively low effect on the QT interval in a rabbit model (i.e. reduced potential for introducing drug-induced QT interval prolongation and appearance of fatal cardiac arrhythmias, torsade de pointes (TdP), in humans). Additionally, the 5-HT₂ antagonistic activity of Compound I suggests that Compound I may have a relatively low risk of extrapyramidal side effects.

The properties outlined above, e.g. binding assays (including alfa-1, DA D1 or D2 receptors), efficacy assays (including DA D1 or D2, or serotonin 5-HT_{2A} receptors), CYP2D6 inhibition and QT-interval may be determined as described in PCT/DK04/000546, cf. in particular the “Example” section page 19-24 in the application text as filed for PCT/DK04/000546.

Further, the inventors have found that Compound I did not induce dystonia when tested in pigs sensitized to haloperidol, indicating that Compound I does not possess EPS (extrapyramidal symptoms) response/liability in humans.

PCT/DK04/000546 describes the following medical uses of Compound I: a disease in the central nervous system, including psychosis, in particular schizophrenia (e.g. positive, negative, and/or depressive symptoms) or other diseases involving psychotic symptoms, such as, e.g., Schizophrenia, Schizophreniform Disorder, Schizoaffective Disorder, Delusional Disorder, Brief Psychotic Disorder, Shared Psychotic Disorder as well other psychotic disorders or diseases that present with psychotic symptoms, e.g. mania in bipolar disorder. Also described is the use of Compound I for treatment of anxiety disorders, affective disorders including depression, sleep disturbances, migraine, neuroleptic-induced parkinsonism, or cocaine abuse, nicotine abuse, alcohol abuse and other abuse disorders.

As indicated in PCT/DK04/000546 a group of compounds structurally related to Compound I, i.e. trans isomers of 3-aryl-1-(1-piperazinyl)indanes substituted in the 2- and/or 3-position of the piperazine ring, has been described in EP 638 073; Bøgesø et al. in J. Med. Chem., 1995, 38, 4380-4392 and Klaus P. Bøgesø in “Drug Hunting, the Medicinal Chemistry of 1-piperazino-3-phenylindans and Related Compounds”, 1998, ISBN 87-88085-10-4I. For example, an enantiomeric pure compound corresponding to formula (I) but differing in that it has an N-methyl group instead of an N-hydrogen on the piperazine has been disclosed in Bøgesø et al. in J. Med. Chem., 1995, 38, 4380-4392, see table 5, compound (−)-38.

None of the above references apart from PCT/DK04/000546 disclose the specific enantiomeric form above (Compound I) or medical use thereof. The trans isomer in the form of the racemate of Compound I is only indirectly disclosed as an intermediate in the synthesis of compound 38 in Bøgesø et al. in J. Med. Chem., 1995, 38, 4380-4392 while medical use of Compound I or its corresponding racemate is not described. Compound I as an intermediate is disclosed in PCT/DK04/000545 (WO05/016900).

### BRIEF DESCRIPTION OF THE DRAWINGS

FIG. 1: Shows an X-ray powder diffractogram of a crystalline hydrogen malate salt of Compound I (obtained using copper K_{α1} radiation (λ=1.5406 Å))

FIG. 2: Shows an X-ray powder diffractogram of a crystalline hydrogen tartrate salt of Compound I (obtained using copper K_{α1} radiation (λ=1.5406 Å))

Further details for the figures are revealed in the Examples below.

### DETAILED DESCRIPTION OF THE INVENTION

The present invention relates to a malate and a tartrate salts of Compound I. The inventors have found that generally it is difficult to obtain solid Compound I in the form of a salt suitable for pharmaceutical formulation, i.e. it has been difficult to find and reproduce salts of Compound I which does have well defined stoichiometry as regards the acid to base ratio and/or salts which are not solvates having water or organic solvents in the crystal. The present invention have overcome these problems by the malate and tartrate salts described herein.

Furthermore, it has been found that an efficient purification of Compound I may be obtained during the manufacture of Compound I by precipitation of the salt of the invention. During the synthesis some cis diastereoisomer of Compound I (i.e. 1-((1S,3S)-6-chloro-3-phenylindan-1-yl)-3,3-dimethylpiperazine) may form as an impurity in the final product. The inventors have found that the final content of cis-isomer may be reduced by precipitation of the salts of Compound I as described herein. Furthermore, purification as regards other impurities detected by HPLC, is significantly enhanced by precipitation of the salts of the invention (cf. Example 10b and 11b).

Accordingly, the invention in one aspect relates to a malate salt, e.g. a L-malate salt, of Compound I, which salt has a well defined stoichiometry as regards the acid to base ratio, e.g. where the ratio between Compound I and the malate is 1:1, e.g. a 1:1 salt of Compound I and L-malate.

A further embodiment of the invention relates to a crystalline malate salt of Compound I, e.g. a crystalline L-malate salt of Compound I, such as a crystalline 1:1 L-malate salt of Compound I. One embodiment of the invention relates to a 1:1 salt of Compound I and malic acid, e.g. L-malic acid, in a substantially anhydrous and solvent free crystalline form.

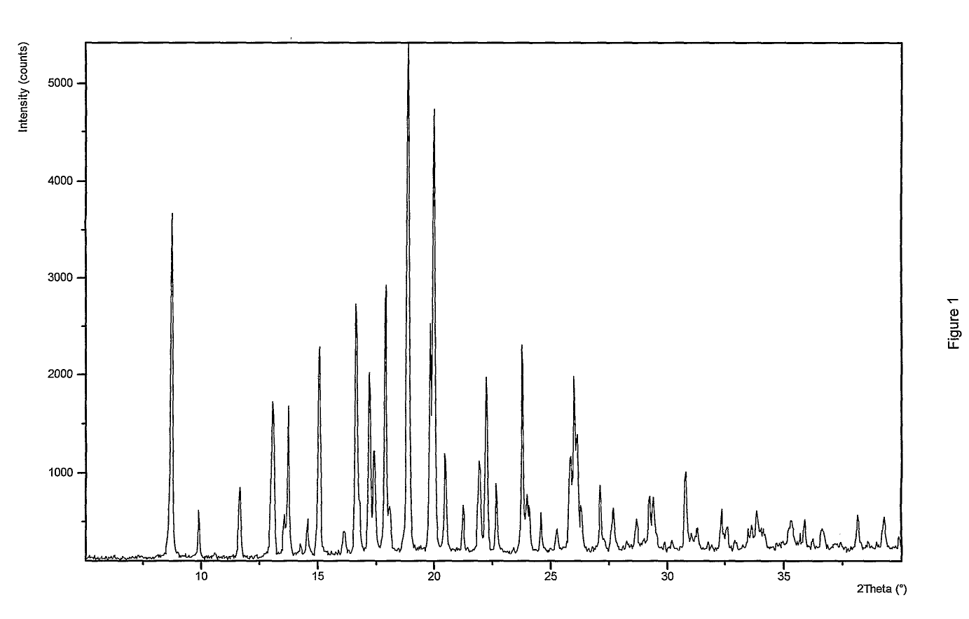

The invention further relates to a crystalline hydrogen L-malate salt (1:1 salt) of Compound I characterized by one or more of:
(i) an X-Ray powder diffractogram as shown in FIG. 1;
(ii) an X-Ray powder diffractogram pattern obtained using copper K_{α1} radiation (λ=1.5406 Å) which shows main peaks at the 2θ-angles given in table 1 below;
(iii) having a DSC (Differential Scanning Calorimetry) trace which shows an endotherm with onset 132-135° C.;
(iv) substantially anhydrous and/or solvent free.

The invention also relates to a tartrate salt, e.g. a L-tartrate salt, of Compound I, which salt has a well defined stoichiometry as regards the acid to base ratio, e.g. where the ratio between Compound I and the tartrate is 1:1, e.g. a 1:1 salt of Compound I and L-tartrate.

One embodiment of the invention relates to a crystalline tartrate salt of Compound I, e.g. a crystalline L-tartrate salt of Compound I, such as a crystalline 1:1 L-tartrate salt of Compound I. The invention also relates to a 1:1 salt of Compound I and tartaric acid, e.g. L-tartaric acid, in a substantially anhydrous and solvent free crystalline form.

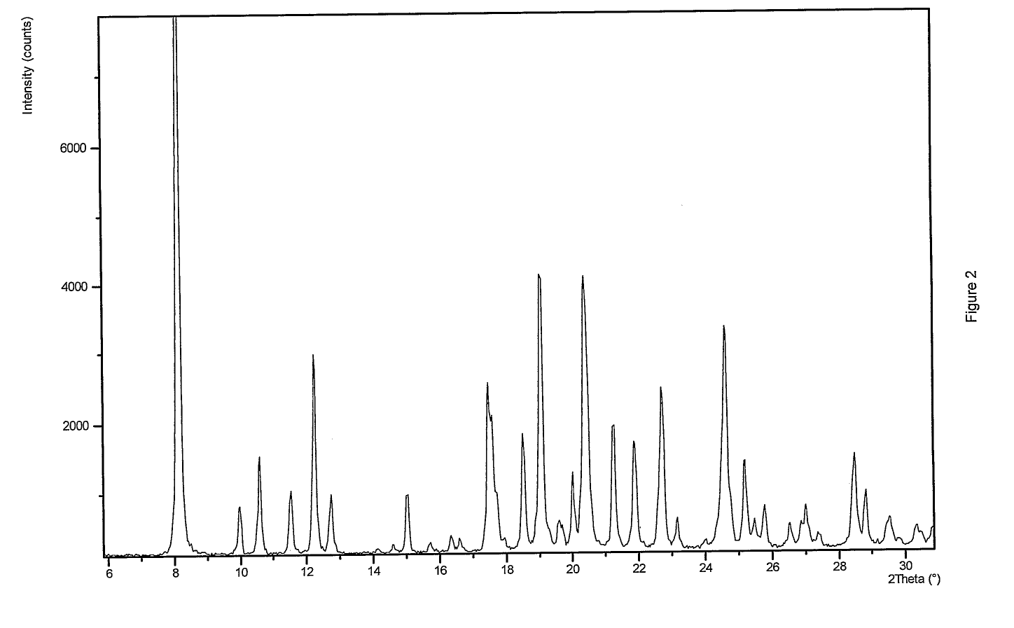

A further embodiment of the invention embodiment relates to crystalline hydrogen L-tartrate salt of Compound I (1:1 salt) characterized by one or more of:
(i) an X-Ray powder diffractogram as shown in FIG. 2;
(ii) an X-Ray powder diffractogram pattern obtained using copper K_{α1} radiation (λ=1.5406 Å) which shows main peaks at the 2θ-angles given in table 1 below;
(iii) having a DSC (Differential Scanning Calorimetry) trace which shows an endotherm with onset 195-199° C.;
(iv) substantially anhydrous and/or solvent free.

TABLE 1
Characteristic reflections (°2 theta) in the X-Ray powder
diffractograms obtained using copper K_{α1} radiation (λ= 1.5406 Å).

| | Characteristic reflexes - main peaks (expressed in |
|---|---|
| Salt | degree of diffraction angle 2θ) |
| Hydrogen malate | 8.7, 9.9, 11.7, 13.1, 13.7, 15.1, 16.7, 18.9, 20.0 |
| Hydrogen tartrate | 8.2, 10.0, 10.6, 11.5, 12.2, 12.7, 15.0, 18.5, 19.1 |

The invention also relates to a salt of the invention having a purity of at least 90%, at least 95% or at least 98% as measured by HPLC (area).

As used herein by expressions like “crystalline form of a specific salt of Compound I characterized by the X-Ray powder diffractogram shown in FIG. (1)” is meant the crystalline form of salt of Compound I in question having an X-ray powder diffractogram substantially similar to FIG. (1), i.e. exhibiting an X-ray powder diffraction pattern substantially as exemplified in that Figure and measured under comparable conditions as described herein or by any comparable method.

That the crystalline salt of the invention is substantially anhydrous and solvent free may e.g. be judged from TGA analysis, e.g. as described in the Examples herein.

Generally, all data herein are understood to be approximate and subject to normal measurement error depending e.g. on the apparatus used and other parameters influencing peak positions and peak intensities.

As indicated above the invention also relates to a crystalline salt of the invention which is not a solvate, i.e. the crystalline salt of the invention does not contain crystal bound solvent molecules. In particular the invention relates to a 1:1 crystalline salt of Compound I and malic acid, e.g. L-malic acid, which crystalline salt is not a solvate. The invention in a further embodiment relates to 1:1 crystalline salt of Compound I and tartaric acid, e.g. L-tartaric acid, which crystalline salt is not a solvate.

In a broad aspect, the invention relates to a crystalline salt of Compound I which salt is a stoichiometrically well defined salt, e.g. where the ratio between Compound I and the respective salt former i.e. acid is 1:1. In one embodiment this crystalline salt is substantially solvent free, e.g. this crystalline salt is both substantially anhydrous and solvent free. In one embodiment this crystalline salt is not a fumarate salt or a maleate salt of Compound I. In a further embodiment this salt of Compound I is not selected from the group consisting of a HCl, a fumarate salt and a maleate salt of Compound I.

Further embodiments of the invention relates to a salt of the invention which is at least 80% crystalline or at least 90% crystalline or at least 95% crystalline and the uses and formulations thereof as described herein for Compound I.

The compound of formula (I) in racemic form may, e.g., be prepared analogously to the methods outlined in EP 638 073, and in Bøgesø et al. J. Med. Chem., 1995, 38, page 4380-4392 followed by optical resolution of the racemic compound by crystallisation of diastereomeric salts thereby obtaining the enantiomer of formula (I), i.e. Compound I. Alternatively, Compound I may be obtained by a method as described in the international patent application PCT/DK04/000546, i.e. from enantiomeric pure V, i.e. compound Va ((1S,3S)-6-chloro-3-phenylindan-1-ol, see below). Compound V has the following formula (V) with cis configuration:

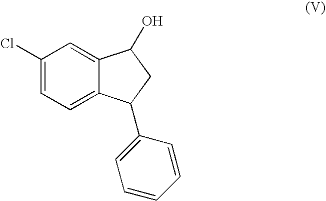

Compound Va has the following formula (Va), i.e. also with cis configuration:

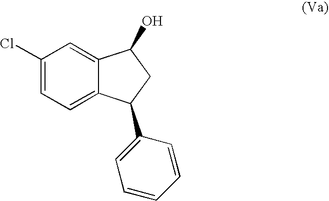

As indicated above during the synthesis some cis diastereoisomer of Compound I (i.e. 1-((1S,3S)-6-chloro-3-phenylindan-1-yl)-3,3-dimethylpiperazine) is formed as an impurity in the final product. The cis form of Compound I may alternatively or additionally, e.g., be removed by precipitation of a suitable salt of the compound of formula Compound I, e.g. HCl or a salt of an organic acid, such as an organic diacid, e.g. a L-(+)-malic salt, L-(+)-tartaric salt, a fumarate salt or a maleate salt of the compound of formula (I), optionally followed by one more re-crystallisations. The cis form of Compound I may also be removed by precipitation of a malate or a tartrate salt of the present invention.

Broadly speaking, the crystalline salts of the invention may be prepared by mixing a solution of either reactant in a solvent, i.e. a suitable single solvent or a suitable mixture of solvents, preferably at room temperature or at elevated temperature, or by adding a solution of either reactant to a solid form of the other reactant and with subsequent precipitation of the crystalline Compound I salt. The term “a solvent” as used herein include both a single solvent or a mixture of different solvents. It is understood that the solvent may comprise water as the case may be, e.g. about 0-20% water. Compound I may be prepared using methods known in the art, such as those described herein. The solvent is preferably an organic solvent, e.g. selected a ketone or an alcohol, e.g. acetone, 2-propanol or ethanol.

The invention also provides a method for the manufacturing of Compound I which process comprises a step of preparing and preferably isolating a salt of the invention, i.e. in particular a malate or a tartrate salt as described herein.

A further aspect of the invention relates to a method for the manufacturing of Compound I, characterised in that the base of Compound I is set free and precipitated to obtain the free base of Compound I in crystalline form, optionally re-crystallised one or more times, and then transferred into a malate or a tartrate salt of the invention I. In one embodiment, the base of Compound I is set free from a crude salt or crude mixture of Compound I. The term crude mixture in this context means that the mixture comprises one or more impurities which it is desired to remove, e.g. the above indicated cis diasteroisomer of Compound I. The crude mixture may be separated directly from the reaction mixture, or the crude reaction mixture may have been subjected to some initial purification. Accordingly, the invention also relates to a malate or a tartrate salt of Compound I obtainable, e.g. obtained, by a process comprising the steps of: (i) crystallising the base of Compound I, and (ii) subsequently forming a malate or a tartrate salt of the invention. The crystalline base of Compound I may be prepared by crystallising, optionally recrystallised one or more times, the base of Compound I from a solvent, e.g. solvent, e.g. of ethyl acetate or heptane or a mixture hereof, e.g. as described in the Examples herein.

The invention also relates to a method for the manufacturing of the following compound of formula II [trans-4-((1R,3S)-6-chloro-3-phenylindan-1-yl)-1,2,2-trimethylpiperazine; Compound II], or a salt thereof,

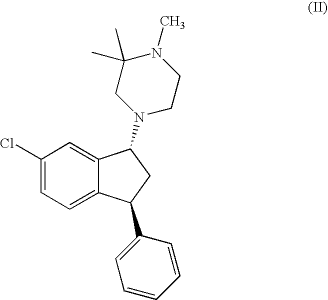

comprising the step of methylation at the secondary amine of Compound I to obtain the free base of Compound II, and alternatively precipitating said compound as a salt, wherein Compound I is produced according to a method of the invention as described herein, i.e. in particular comprising a step in which Compound I is precipitated as a salt of the invention. The synthesis of Compound II from Compound I is described in WO05/016900. The salt of Compound II may, e.g., be a succinate or a malonate salt, e.g. a hydrogen succinate salt or a hydrogen malonate salt as described in WO05/016900. In further embodiments, Compound II or a salt thereof may subsequently by formulated into a pharmaceutical composition.

The properties of Compound I indicate that it will be particularly useful as a pharmaceutical. Accordingly, the present invention further relates to a pharmaceutical composition of a salt of the invention. The invention also relates to the medical use of such a salt and composition, such as for the treatment of a disease in the central nervous system, including psychosis, in particular schizophrenia or other diseases involving psychotic symptoms, such as, e.g., Schizophrenia, Schizophreniform Disorder, Schizoaffective Disorder, Delusional Disorder, Brief Psychotic Disorder, Shared Psychotic Disorder as well other psychotic disorders or diseases that present with psychotic symptoms, e.g. mania in bipolar disorder.

The present invention also relates to use of a salt of the invention for treatment of a disease selected from the group consisting of anxiety disorders, affective disorders including depression, sleep disturbances, migraine, neuroleptic-induced parkinsonism, cocaine abuse, nicotine abuse, alcohol abuse and other abuse disorders.

The invention also relates to a method of treating Schizophreniform Disorder, Schizoaffective Disorder, Delusional Disorder, Brief Psychotic Disorder, Shared Psychotic Disorder or mania in bipolar disorder, comprising administering a therapeutically effective amount of a salt of the invention.

A further embodiment of the invention relates to a method of treating positive symptoms of schizophrenia comprising administering a therapeutically effective amount of a salt of the invention. Another embodiment of the invention relates to a method of treating negative symptoms of schizophrenia comprising administering a therapeutically effective amount of a salt of the invention. A further embodiment of the invention relates to a method of treating depressive symptoms of schizophrenia comprising administering a therapeutically effective amount of a salt of the invention.

A further aspect of the invention relates to a method of treating mania and/or maintenance of bipolar disorder comprising administering a therapeutically effective amount of a salt of the invention.

The invention further relates to a method of treating substance abuse, e.g. nicotine, alcohol or cocaine abuse, comprising administering a therapeutically effective amount of a salt of the invention.

In the present context, in particular for the pharmaceutical uses, it is understood that when specifying the enantiomer form as done in formula (I) for Compound I, then the compound is relatively stereochemically pure, preferably the enantiomeric excess is of at least 70%, and more preferably at least 80% (80% enantiomeric excess means that the ratio of I to its enantiomer is 90:10 in the mixture in question) at least 90%, at least 96%, or preferably at least 98%. In a preferred embodiment, the diastereomeric excess of Compound I is at least 90% (90% diastereomeric excess means the ratio of Compound I to cis-1-((1S,3S)-6-chloro-3-phenylindan-1-yl)-3,3-dimethylpiperazine is 95:5), at least 95%, at least 97%, or at least 98%.

A further aspect of the invention relates to a method of treatment as described herein, wherein the patient treated with a salt of the invention is also treated with at least one other medicament. A particular relevant embodiment in this connection, is treatment with other medicaments being metabolised by CYP2D6. In a suitable embodiment, the other medicament is an antipsychotic. Accordingly, one embodiment relates to the use a salt of the invention for treating a patient suffering from schizophrenia or other psychoses who is also treated with other medicament(s), e.g. where this other medicament is an antipsychotic. In another embodiment, the invention relates to the use of a salt of the invention for treating a patient suffering from schizophrenia or other psychoses who is a substance abuser, e.g. of alcohol or narcotics.

The compound, salt or composition of the invention may be administered in any suitable way e.g. orally, buccal, sublingual or parenterally, and the compound or salt may be presented in any suitable form for such administration, e.g. in the form of tablets, capsules, powders, syrups or solutions or dispersions for injection. In one embodiment, the compound or salt of the invention are administered in the form of a solid pharmaceutical entity, suitably as a tablet or a capsule.

Methods for the preparation of solid pharmaceutical preparations are well known in the art. Tablets may thus be prepared by mixing the active ingredient with ordinary adjuvants, fillers and diluents and subsequently compressing the mixture in a convenient tabletting machine. Examples of adjuvants, fillers and diluents comprise corn starch, lactose, talcum, magnesium stearate, gelatine, lactose, gums, and the like. Any other adjuvant or additive such as colourings, aroma, preservatives, etc. may also be used provided that they are compatible with the active ingredients.

Solutions for injections may be prepared by dissolving a salt of the invention and possible additives in a part of the solvent for injection, preferably sterile water, adjusting the solution to desired volume, sterilisation of the solution and filling in suitable ampules or vials. Any suitable additive conventionally used in the art may be added, such as tonicity agents, preservatives, antioxidants, solubilising agents etc.

The daily dose of the compound of formula (I) above, calculated as the free base, is suitably between 1.0 and 160 mg/day, more suitable between 1 and 100 mg, e.g. preferably between 2 and 55 mg.

As indicated above the invention in particular relates to:
a salt of the invention
a pharmaceutical compositions as described herein comprising a salt of the invention;
a medical use of such salt of the invention as described herein for Compound I;
   wherein Compound I is having an enantiomeric excess of at least 60% (60% enantiomeric excess means that the ratio of Compound I to its enantiomer is 80:20 in the mixture in question), at least 70%, at least 80%, at least 85%, at least 90%, at least 96%, preferably at least 98%.

One embodiment relates to a salt of the invention or pharmaceutical composition of the invention and the uses as described herein, wherein Compound I is having a diastereomeric excess of at least 10% (10% diastereomeric excess means that the ratio of Compound I to the cis-(1S,3S) diastereoisomer is 55:45 in the mixture in question), at least 25%, at least 50%, at least 70%, at least 80%, at least 90%, at least 95%, at least 97%, preferably at least 98%.

The term “treatment” in connection with a disease as used herein also includes prevention as the case may be. The term “disease” as used herein also includes a disorder as the case may be.

The invention will be illustrated in the following non-limiting examples.

### EXAMPLES

### Analytical Methods

The enantiomeric excess of Compound (Va) in Example 1 is determined by chiral HPLC using a CHIRALCEL® OD column, 0.46 cm ID×25 cm L, 10 μm at 40° C. n-Hexan/ethanol 95:5 (vol/vol) is used as mobile phase at a flow rate of 1.0 ml/min, detection is performed using a UV detector at 220 nm.

The enantiomeric excess of Compound I is determined by fused silica capillary electrophoresis (CE) using the following conditions: Capillar: 50 μm ID×48.5 cm L, run buffer: 1.25 mM β cyclo dextrin in 25 mM sodium dihydrogen phosphate, pH 1.5, voltage: 16 kV, temperature: 22° C., injection: 40 mbar for 4 seconds, detection: column diode array detection 195 nm, sample concentration: 500 μg/ml. In this system, Compound I has a retention time of approximately 10 min, and the other enantiomer has a retention time of approximately 11 min.

¹H NMR spectra is recorded at 500.13 MHz on a Bruker Avance DRX500 instrument or at 250.13 MHz on a Bruker AC 250 instrument. Chloroform (99.8% D) or dimethyl sulfoxide (99.8% D) is used as solvents, and tetramethylsilane (TMS) is used as internal reference standard.

The purity of Compound I is determined by HPLC (e.g. also the cis/trans ratio) using a Luna C18(2) 150*4.6 mm (3 μm) column at 40° C. The mobile phase is phosphate buffer pH7.4/acetonitril 40/60, run time 60 min, and after 32 min a gradient of acetonitril/water 90/10 is applied. Detection is performed using a UV detector at 220 nm.

The cis/trans ratio of Compound I and key intermediates is determined using ¹H NMR, e.g. as described in Bøgesø et al., J. Med. Chem. 1995, 38, 4380-4392 (page 4388, right column). Generally, a content of approximately 1% of the undesired isomer can be detected by NMR.

The Melting points are measured using Differential Scanning Calorimetry (DSC). The equipment is a TA-Instruments DSC-Q1000 calibrated at 5°/min to give the melting point as onset value. About 2 mg of sample is heated 5°/min in a loosely closed pan under nitrogen flow.

Thermo gravimetric analysis (TGA) used for estimation of solvent/water content of dried material is performed using a TA-instruments TGA-Q500. 1-10 mg sample is heated 10°/min in an open pan under nitrogen flow.

X-Ray powder diffractograms were measured on a PANalytical X'Pert PRO X-Ray Diffractometer using CuK_{α1} radiation. The samples were measured in reflection mode in the 2θ-range 5-40° using an X'celerator detector.

Optical rotation is measured on a polarimeter, Perkin Elmer model 241.

### Synthesis

### Example 1 Synthesis of (1S,3S)-6-chloro-3-phenylindan-1-ol (Va) by use of chiral chromatography

Racemic cis-6-chloro-3-phenylindan-1-ol (V) (prepared as described in PCT/DK04/000546, i.e. adapting the method described in Bøgesø et al. J. Med. Chem. 1995, 38, 4380-4392 using ethanol as solvent, and performing the reaction at approximately 0° C.) (492 grams) is resolved by preparative chromatography, using a CHIRALPAK® AD column, 10 cm ID×50 cm L, 10 μm at 40° C. Methanol is used as mobile phase at a flow rate of 190 ml/min, detection is performed using a UV detector at 287 nm. The racemic alcohol (V) is injected as a 50,000 ppm solution in methanol; 90 ml is injected with intervals of 28 min. All the fractions, which contain the title compound with more than 98% enantiomeric excess, are combined and evaporated to dryness using a rotary evaporator, followed by drying in vacuo at 40° C. Yield 220 grams as a solid. Elemental analysis and NMR conform to the structure, the enantiomeric excess is higher than 98% according to chiral HPLC, [α]_{D}²⁰+44.5° (c=1.0, methanol).

### Example 2 Synthesis of (1S,3S)-3,5-dichloro-1-phenylindan

Cis-(1S,3S)-6-chloro-3-phenylindan-1-ol (Va) (204 grams) obtained as described in Example 1 is dissolved in THF (1500 ml) and cooled to −5° C. Thionyl chloride (119 grams) is added dropwise as a solution in THF (500 ml) over a period of 1 h. The mixture is stirred at room temperature over night. Ice (100 g) is added to the reaction mixture. When the ice has melted the water phase (A) and the organic phase (B) are separated, and the organic phase B is washed twice with aqueous saturated sodium bicarbonate (200 ml). The aqueous sodium bicarbonate phases are combined with water phase A, adjusted to pH 9 with sodium hydroxide (28%), and used to wash the organic phase B once again. The resulting aqueous phase (C) and the organic phase B are separated, and the aqueous phase C is extracted with ethyl acetate. The ethyl acetate phase is combined with the organic phase B, dried with magnesium sulphate, and evaporated to dryness using a rotary evaporator, giving the title compound as an oil. Yield 240 grains, which is used directly in the example 5a. Cis/trans ratio 77:23 according to NMR.

### Example 3 Synthesis of 3,3-dimethylpiperazin-2-one

Potassium carbonate (390 grams) and ethylene diamine (1001 grams) are stirred with toluene (1.50 l). A solution of ethyl 2-bromoisobutyrate (500 grams) in toluene (750 ml) is added. The suspension is heated to reflux over night, and filtered. The filter cake is washed with toluene (500 ml). The combined filtrates (volume 4.0 l) are heated on a water bath and distilled at 0.3 atm. using a Claisen apparatus; first 1200 ml distillate is collected at 35° C. (the temperature in the mixture is 75° C.). More toluene is added (600 ml), and another 1200 ml distillate is collected at 76° C. (the temperature in the mixture is 80° C.). Toluene (750 ml) is added again, and 1100 ml of distillate is collected at 66° C. (temperature in the mixture 71° C.). The mixture is stirred on an ice bath and inoculated, whereby the product precipitates. The product is isolated by filtration, washed with toluene, and dried over night in a vacuum oven at 50° C. Yield 171 g (52%) of 3,3-dimethylpiperazin-2-one. NMR consistent with structure.

### Example 4 Synthesis of 2,2-dimethylpiperazine

A mixture of 3,3-dimethylpiperazin-2-one (8.28 kg, 64.6 mol, large scale preparation analogous to the preparation described in to Example 3) and tetrahydrofuran (THF) (60 kg) is heated to 50-60° C. giving a slightly unclear solution. THF (50 kg) is stirred under nitrogen, and LiAlH₄ (250 g, in a soluble plastic bag) is added, which gives a slow evolution of gas. After gas evolution has ceased, more LiAlH₄ is added (a total of 3.0 kg, 79.1 mol, is used), and the temperature rises from 22° C. to 50° C. because of an exoterm. The solution of 3,3-dimethylpiperazin-2-one is added slowly over 2 hours at 41-59° C. The suspension is stirred for another hour at 59° C. (jacket temperature 60° C.). The mixture is cooled, and water (3 l) is added over two hours, keeping the temperature below 25° C. (it is necessary to cool with a jacket temperature of 0° C.). Then aqueous sodium hydroxide (15%, 3.50 kg) is added over 20 minutes at 23° C., cooling necessary. More water (9 l) is added over half an hour (cooling necessary), and the mixture is stirred over night under nitrogen. Filter agent Celite (4 kg) is added, and the mixture is filtered. The filter cake is washed with THF (40 kg). The combined filtrates are concentrated in the reactor until the temperature in the reactor is 70° C. (distillation temperature 66° C.) at 800 mbar. The remanence (12.8 kg) is further concentrated on a rotary evaporator to approximately 10 l. Finally, the mixture is fractionally distilled at atmospheric pressure, and the product is collected at 163-4° C. Yield 5.3 kg (72%). NMR complies with the structure.

### Example 5a Synthesis of trans-1-((1R,3S)-6-chloro-3-phenylindan-1-yl)-3,3-dimethylpiperazinium (Compound I) hydrogen maleate salt

Cis-(1S,3S)-3,5-dichloro-1-phenylindan (240 g) is dissolved in butan-2-one (1800 ml). Potassium carbonate (272 g) and 2,2-dimethyl piperazine (prepared as described in Example 4) (113 g) are added and the mixture is heated at reflux temperature for 40 h. To the reaction mixture is added diethyl ether (2 l) and hydrochloric acid (1M, 6 l). The phases are separated, and pH in the water phase is lowered from 8 to 1 with concentrated hydrochloric acid. The water phase is used to wash the organic phase once again in order to ensure, that all product is in the water phase. Sodium hydroxide (28%) is added to the water phase until pH is 10, and the water phase is extracted twice with diethyl ether (2 l). The diethyl ether extracts are combined, dried with sodium sulphate, and evaporated to dryness using a rotary evaporator. Yield 251 grams of free base of Compound I as an oil. Cis/trans ratio, 18:82 according to NMR. The crude oil (ca. 20 grams) was further purified by flash chromatography on silicagel (eluent: ethyl acetate/ethanol/triethylamine 90:5:5) followed by evaporation to dryness on a rotary evaporator. Yield 12 grams of free base of Compound I as an oil (cis/trans ratio, 10:90 according to NMR).

The oil is dissolved in ethanol (100 ml), and to this solution is added a solution of maleic acid in ethanol to pH 3. The resulting mixture is stirred at room temperature for 16 hours, and the formed precipitate is collected by filtration. The volume of ethanol is reduced and another batch of precipitate is collected. Yield 3.5 gram solid, i.e. Compound I hydrogen maleate salt (no cis isomer is detected according to NMR) of the title compound.

Enantiomeric excess according to CE is >99%. Melting point 175-178° C. NMR complies with the structure.

### Example 5b Synthesis of trans-1-((1R,3S)-6-chloro-3-phenylindan-1-yl)-3,3-dimethylpiperazinium (Compound I) hydrogen chloride salt

Cis-(1S,3S)-3,5-dichloro-1-phenylindan (large scale preparation analogous to the preparation described in example 2) (50.9 kg) is dissolved in MIBK (248 kg). Potassium carbonate (56.8 kg) and 2,2-dimethyl piperazine (29.6 kg) are added and the mixture is heated to 100° C. temperature for 8 hour. The reaction mixture is cooled to room temperature before insoluble inorganic material is removed by filtration. The filtrate is subsequent washed with water (520 l), the phases are separated and pH of the organic phase is adjusted to a value between 3-6 by slow addition of hydrogen chloride (15.4 kg 37% aqueous solution), during the addition the product separates. The product is filtered on a nutsche, and the filter cake is washed by MIBK (100 kg) and cyclohexane (80 kg). The product is dried at 50° C. and 0.05 bar for 12 hours.

Yield: 40 kg. Compound I (no cis-isomer is detected according to NMR-analysis). Enantiomeric excess according to CE is >99%. NMR spectrum complies with the structure.

### Example 6a Synthesis of the Free Base of Compound I from a Hydrogen Maleate Salt

A mixture of trans-1-((1R,3S)-6-chloro-3-phenylindan-1-yl)-3,3-dimethylpiperazinium hydrogen maleate (9.9 grams), concentrated aqueous ammonia (100 ml), brine (150 ml) and ethyl acetate (250 ml) is stirred at room temperature for 30 min. The phases are separated, and the aqueous phase is extracted with ethyl acetate once more. The combined organic phases are washed with brine, dried over magnesium sulphate, filtered and evaporated to dryness in vacuo. Yield 7.5 grams of Compound I as an oil, which may solidify on standing. NMR complies with the structure.

### Example 6b Synthesis of the Free Base of Compound I from a Hydrogen Chloride Salt

The free base of Compound I was prepared as described in example 6a by the use of trans-1-((1R,3S)-6-chloro-3-phenylindan-1-yl)-3,3-dimethylpiperazinium hydrogen chloride as substitute for trans-1-((1R,3S)-6-chloro-3-phenylindan-1-yl)-3,3-dimethylpiperazinium hydrogen maleate.

Yield of Compound I was 9.0 grams starting from 10.2 grams of trans-1-((1R,3S)-6-chloro-3-phenylindan-1-yl)-3,3-dimethylpiperazinium hydrochloride.

### Example 7a Preparation of Crystalline Base of Compound I from 6a

Compound I (9.0 grams obtained as described in Example 6a) was dissolved in ethyl acetate (30 ml), and heptane (75 ml) was subsequently added to the solution. The solution was left with stirring for 4-16 hours. In some cases crystallisation was observed, and the crystalline material was collected by filtration. In other cases, crystallisation was not observed, and a part of the solvent was removed by distillation. The distillation was stopped when distillation temperature changed from the boiling point of ethyl acetate to the boiling point of heptane. The remaining solution was left to cool to room temperature at ambient temperature and prior to filtration on a water/ice bath. The crystallisation could be initiated by scratching with a glass spatula or by seeding. The crystalline Compound I was isolated by filtration. Yield 6.8 grams (74%). NMR complies with the structure. Melting point: 92.4° C. (DSC onset temperature), enantiomeric excess according to CE is >99%.

### Example 7b Preparation of crystalline base of Compound I from 6b

Prepared as described in example 7a starting with 9.0 grams of crude base. Yield 6.8 grams. Melting point 92.3° C. (DSC onset temperature) and enantiomeric excess determined by CE is >99%.

### Example 8 Characterisation of the Crystalline Base of Compound I

The crystalline base of Compound I obtained by a method as described in Example 7a and 7b had the X-ray powder diffractogram (XRPD) shown in FIG. 1 and was characterized by the following reflections (peaks) in the X-Ray powder diffractogram as measured using CuKα₁ radiation at 2-theta angles: 6.1, 11.1, 12.1, 16.2, 16.8, 18.3, 18.6, 20.0. The crystalline base further had a DSC thermogram corresponding to that of FIG. 2 and a DSC trace showing an endotherm with onset about 91-93° C. The crystalline based obtained was anhydrous and solvent free as judged from TGA analysis.

### Example 9 Synthesis of trans-1-((1R,3S)-6-chloro-3-phenylindan-1-yl)-3,3-dimethylpiperazinium (Compound I) Fumarate Salt

A solution of trans-1-((1R,3S)-6-chloro-3-phenylindan-1-yl)-3,3-dimethylpiperazine (obtained as described in Example 6a) (1 g) is dissolved in acetone (100 mL). To this solution is added a solution of fumaric acid in ethanol until pH of the resulting solution is 4. The resulting mixture is cooled in an ice bath for 1.5 hours whereby a precipitate is formed. The solid compound is collected by filtration. The compound was dried in vacuo giving a white solid compound (1.0 g). Enantiomeric excess is >99%. Melting point 193-196° C. NMR complies with the structure.

### Example 10a Synthesis of trans-1-((1R,3S)-6-chloro-3-phenylindan-1-yl)-3,3-dimethylpiperazinium (Compound I) L-tartrate salt

trans-1-((1R,3S)-6-Chloro-3-phenylindan-1-yl)-3,3-dimethylpiperazine (obtained as described in example 6b) (2 grams) is dissolved in ethanol (20 ml). L-tartaric acid (0.88 grams) is added at 60° C. When precipitation is detected the reaction mixture is cooled to below room temperature and kept at this temperature for 1 hour. The precipitate of trans-1-((1R,3S)-6-chloro-3-phenylindan-1-yl)-3,3-dimethylpiperazinium L-tartrate is filtered off and the filter cake is washed with ethanol (5 ml). The filter cake is sucked free of most of the solvent, and the product is dried “in vacuo” at 50° C. over-night. As TGA analysis only show a weight loss up to 0.5% the product is regarded substantially free of residual solvent or water.

Yield 2.50 grams (87%). Enantiomeric excess is >99%.

### Example 10b Synthesis of trans-1-((1R,3S)-6-chloro-3-phenylindan-1-yl)-3,3-dimethylpiperazinium (Compound I) L-tartrate salt

Compound I (2.4 grams of crude oil obtained as described in Example 5a, purity as determined by HPLC: 73% area, cis/trans ratio of 17/73) is dissolved in ethanol (20 ml). L-tartaric acid (1.06 grams) is added at 60° C. When precipitation is detected the reaction mixture is cooled to below room temperature and kept at this temperature for 1 hour. The precipitate of trans-1-((1R,3S)-6-chloro-3-phenylindan-1-yl)-3,3-dimethylpiperazinium L-tartrate is filtered off and the filter cake is washed with ethanol (10 ml). The filter cake is sucked free of most of the solvent, and the product is dried “in vacuo” at 50° C. over-night. Yield 2.01 grams. Further purification by recrystallisation from ethanol.

Yield: 1.1 gram NMR complies with the structure. As TGA analysis show no weight loss before degradation occurs, the product is regarded substantially free of residual solvent or water. HPLC Purity (area %): 96%, content of the cis-isomer: 4%. Enantiomeric excess according to CE is >99%.

### Example 11a Synthesis of trans-1-((1R,3S)-6-chloro-3-phenylindan-1-yl)-3,3-dimethylpiperazinium (Compound I) L-malate salt

trans-1-((1R,3S)-6-Chloro-3-phenylindan-1-yl)-3,3-dimethylpiperazine (obtained as described in Example 6b) (2 grams) is dissolved in 2-propanol (20 ml). L-malic acid (0.79 grams) is added at 60° C. When precipitation is detected the reaction mixture is cooled to below room temperature and kept at this temperature for 1 hour. The precipitate of trans-1-((1R,3S)-6-chloro-3-phenylindan-1-yl)-3,3-dimethylpiperazinium L-malate is filtered off and the filter cake is washed with 2-propanol (5 ml). The filter cake is sucked free of most of the solvent before the product is dried “in vacuo” at 50° C. over-night. As TGA analysis only show a weight loss up to 0.5% the product is regarded substantially free of residual solvent or water.

Yield 2.38 grams (85%). Enantiomeric excess is >99%.

### Example 11b Synthesis of trans-1-((1R,3S)-6-chloro-3-phenylindan-1-yl)-3,3-dimethylpiperazinium (Compound I) L-malate salt

Compound I (2.25 grams of crude oil obtained as described in Example 5a, purity as determined by HPLC: 73% area, cis/trans ratio of 17/73) is dissolved in 2-propanol (22.5 ml). L-malic acid (0.89 grams) is added at 60° C. When precipitation is detected the reaction mixture is cooled to below room temperature and kept at this temperature for 1 hour. The precipitate of trans-1-((1R,3S)-6-chloro-3-phenylindan-1-yl)-3,3-dimethylpiperazinium L-malate is filtered off and the filter cake is washed with 2-propanol (10 ml). The filter cake is sucked free of most of the solvent before the product is dried “in vacuo” at 50° C. over-night. As TGA analysis show no weight loss before degradation occurs the product is regarded substantially free of residual solvent or water.

Yield 1.55 grams NMR complies with the structure. HPLC Purity (area %): 96%, content of the cis-isomer: 2%. Enantiomeric excess according to CE is >99%.

### Example 12 Characterisation of the L-malate and L-tartrate Salts

The L-malate salt and the L-tartrate salt obtained by the methods described above in Example 10 and 11 are crystalline and stoichiometrically well defined as 1:1 salts which means they are hydrogen L-malate and hydrogen L-tartrate respectively.

FIG. 1 shows an X-ray powder diffractogram of the crystalline hydrogen L-malate salt of Compound I. FIG. 2 Shows an X-ray powder diffractogram of the crystalline hydrogen L-tartrate salt of Compound I.

The salts are crystalline solids. The solubility of the malate salt is 0.8 mg/ml and the solubility of the tartrate salt is 0.5 mg/ml.

1. A malate salt of Compound I, wherein Compound I is of formula (I):

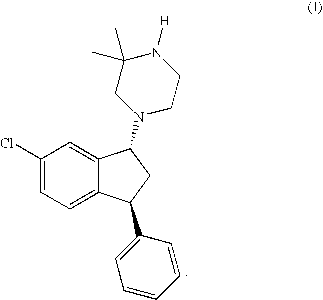

2. The salt of claim 1, wherein the salt is an L-malate salt of Compound I.

3. The salt of claim 1, wherein the salt has an acid to base ratio of 1:1 Compound I to malate.

4. The salt of claim 1, wherein the salt is crystalline.

5. The salt of claim 1, wherein the salt is a crystalline L-malate salt having an acid to base ratio of 1:1 Compound I to L-malate.

6. The salt of claim 5, wherein the salt is characterised by an X-Ray powder diffractogram corresponding to that of FIG. 1.

7. The salt of claim 5, wherein the salt is characterized by an X-Ray powder diffractogram obtained using CuK_{α1} radiation (X=1.5406 Å) and showing peaks at 2θ-angles of: 8.7, 9.9, 11.7, 13.1, 13.7, 15.1, 16.7, 18.9, and 20.0.

8. The salt of claim 1, wherein the salt is characterized by having a DSC trace showing an endotherm with an onset at about 132° C. to about 135° C.

9. A tartrate salt of Compound I, wherein Compound I is of formula (I):

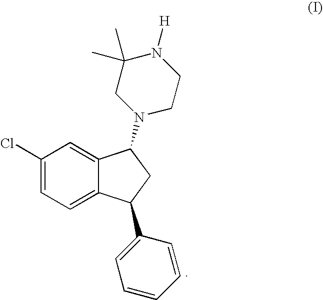

10. The salt of claim 9, wherein the salt is an L-tartrate salt of Compound I.

11. The salt of claim 9, wherein the salt has an acid to base ratio of 1:1 Compound I to tartrate.

12. The salt of claim 9, wherein the salt is crystalline.

13. The salt of claim 9, wherein the salt is a crystalline L-tartrate salt having an acid to base ratio of 1:1 Compound I to L-tartrate.

14. The salt of claim 13, wherein the salt is characterized by an X-Ray powder diffractogram corresponding to that of FIG. 2.

15. The salt of claim 13, wherein the salt is characterized by an X-Ray powder diffractogram obtained using CuK_{α1} radiation (λ=1.5406 Å) and showing peaks at 2θ-angles of: 8.2, 10.0, 10.6, 11.5, 12.2, 12.7, 15.0, 18.5, and 19.1

16. The salt of claim 9, wherein the salt is characterized by having a DSC trace showing an endotherm with an onset at about 195° C. to about 199° C.

17. The salt of claim 1, characterized in that it is at least about 80% crystalline.

18. The salt of claim 1, wherein the salt is a substantially anhydrous crystalline salt of Compound I.

19. The salt of claim 18, wherein the substantially anhydrous crystalline salt is solvent free.

20. The salt of claim 1, wherein Compound I of the salt has a purity of at least about 95% or at least about 98% as measured by HPLC.

21. A pharmaceutical composition comprising the salt of claim 1 and a pharmaceutically acceptable adjuvant, diluent, carrier, additive, or combination thereof.

22. The pharmaceutical composition according to claim 21, wherein an enantiomeric excess of Compound I is at least about 70%, at least about 80%, least about 90%, at least about 96%, or at least about 98%.

23. The pharmaceutical composition according to claim 21, wherein a diastereomeric excess of Compound I is at least about 80%, least about 90%, at least about 96%, or at least about 98%.

24. The salt of claim 1 for use in medicine.

25. A pharmaceutical composition comprising the salt of claim 1 for the treatment of a disease selected from the group consisting of a disease involving a psychotic symptom, an anxiety disorder, an affective disorder, a sleep disturbance disorder, migraine, neuroleptic-induced parkinsonism, and an abuse disorder.

26. A pharmaceutical composition comprising the salt of claim 1 for the treatment of schizophrenia or another psychotic disorder.

27. A pharmaceutical composition comprising the salt of claim 1 for the treatment of a disease selected from the group consisting of Schizophrenia, Schizophreniform Disorder, Schizoaffective Disorder, Delusional Disorder, Brief Psychotic Disorder, Shared Psychotic Disorder, and mania in bipolar disorder.

28. A pharmaceutical composition comprising the salt of claim 1 for the treatment of one or more symptom of schizophrenia, wherein the symptom of schizophrenia is a positive symptom, a negative symptom, a depressive symptom, or a combination thereof.

29. A method for the treatment of a disease selected from the group consisting of a disease involving a psychotic symptom, schizophrenia, an anxiety disorder, an affective disorder, a sleep disturbance disorder, migraine, neuroleptic-induced parkinsonism, or an abuse disorder, the method comprising administering a therapeutically effective amount of the salt of claim 1.

30. The method of claim 29, wherein the disease is schizophrenia or a disease involving a psychotic symptom.

31. The method of claim 30, wherein the schizophrenia comprises a positive symptom, a negative symptom, a depressive symptom, or a combination thereof.

32. A method for the treatment of a disease selected from the group consisting of Schizophrenia, Schizophreniform Disorder, Schizoaffective Disorder, Delusional Disorder, Brief Psychotic Disorder, Shared Psychotic Disorder, and mania in bipolar disorder, the method comprising administering a therapeutically effective amount of the salt of claim 1.

33. The pharmaceutical composition of claim 15, wherein a patient treated with the pharmaceutical composition is also treated with at least one other medicament, wherein Compound I is absent from the other medicament.

34. A method of the manufacturing the salt of claim 1, wherein the method comprises preparing and isolating the salt.

35. A method of manufacturing a salt of Compound I: 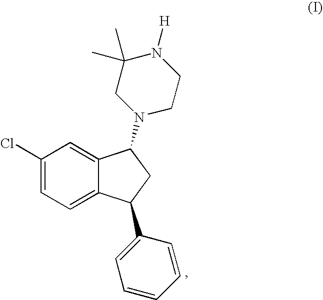
the method comprising setting free a base of Compound I;
precipitating the base of Compound I in crystalline form; optionally recrystallizing the crystalline base of Compound I;
and transferring the crystalline base of Compound I into a salt of Compound I, wherein the salt is as defined in claim 1.

36. The method of claim 35, wherein the base of Compound I is set free from a crude salt or a crude mixture of Compound I.

37. The method of claim 34, further comprising making a pharmaceutical composition comprising the salt of Compound I and a pharmaceutically acceptable adjuvant, diluent, carrier, additive, or combination thereof.

38. The method of claim 34 further comprising crystallizing a base of Compound I by precipitating the base of Compound I from a solvent; and separating the solvent from the obtained crystalline base of Compound I.

39. A method of manufacturing a compound of formula II: 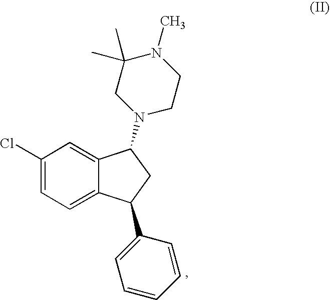
the method comprising methylating a secondary amine of Compound I: 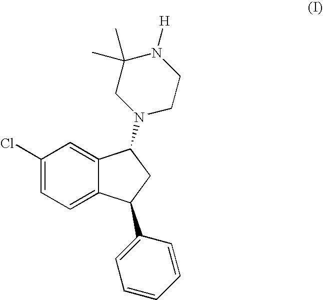
to obtain a free base of the compound of formula II; wherein Compound I is produced by the method of claim 34.

40. The method of claim 39, further comprising precipitating the compound of formula II as a salt.

41. The method of claim 40, wherein the salt is a succinate salt or a malonate salt.

42. The method of claim 39, further comprising making a pharmaceutical composition comprising the compound of formula II or a salt thereof and a pharmaceutically acceptable adjuvant, diluent, carrier, additive, or combination thereof.

43. The pharmaceutical composition of claim 25, wherein the affective disorder is depression.

44. The pharmaceutical composition of claim 25, wherein the abuse disorder is cocaine abuse, nicotine abuse, or alcohol abuse.

45. The method of claim 29, wherein the affective disorder is depression.

46. The method of claim 29, wherein the abuse disorder is cocaine abuse, nicotine abuse, or alcohol abuse.

47. The method of claim 38, wherein the solvent comprises heptane.

48. A pharmaceutical composition comprising the salt of claim 9.

49. The pharmaceutical composition according to claim 48, wherein an enantiomeric excess of Compound I is at least about 70%, at least about 80%, at least about 90%, at least about 96%, or at least about 98%.

50. The pharmaceutical composition according to claim 48, wherein a diastereomeric excess of Compound I is at least about 80%, least about 90%, at least about 96%, or at least about 98%.

51. The salt of claim 9 for use in medicine.

52. A pharmaceutical composition comprising the salt of claim 9 for the treatment of a disease selected from the group consisting of a disease involving a psychotic symptom, an anxiety disorder, an affective disorder, a sleep disturbance disorder, migraine, neuroleptic-induced parkinsonism, and an abuse disorder.

53. The use of claim 49, wherein the affective disorder is depression.

54. The pharmaceutical composition of claim 52, wherein the abuse disorder is cocaine abuse, nicotine abuse, or alcohol abuse.

55. The pharmaceutical composition of claim 52, wherein a patient treated with the pharmaceutical composition is also treated with at least one other medicament, wherein Compound I is absent from the other medicament.

56. A pharmaceutical composition comprising the salt of claim 9 for the treatment of schizophrenia or another psychotic disorder.

57. A pharmaceutical composition comprising the salt of claim 9 for the treatment of a disease selected from the group consisting of Schizophrenia, Schizophreniform Disorder, Schizoaffective Disorder, Delusional Disorder, Brief Psychotic Disorder, Shared Psychotic Disorder, and mania in bipolar disorder.

58. A pharmaceutical composition comprising the salt of claim 9 for the treatment of one or more symptom of schizophrenia, wherein the symptom of schizophrenia is a positive symptom, a negative symptom, a depressive symptom, or a combination thereof.

59. A method for the treatment of a disease selected from the group consisting of a disease involving a psychotic symptom, schizophrenia, an anxiety disorder, an affective disorder, a sleep disturbance disorder, migraine, neuroleptic-induced parkinsonism, or an abuse disorder, the method comprising administering a therapeutically effective amount of the salt of claim 9.

60. The method of claim 59, wherein the affective disorder is depression.

61. The method of claim 59, wherein the abuse disorder is cocaine abuse, nicotine abuse, or alcohol abuse.

62. The method of claim 59, wherein the disease is schizophrenia or a disease involving a psychotic symptom.

63. The method of claim 62, wherein the schizophrenia comprises a positive symptom, a negative symptom, a depressive symptom, or a combination thereof.

64. A method for the treatment of a disease selected from the group consisting of Schizophrenia, Schizophreniform Disorder, Schizoaffective Disorder, Delusional Disorder, Brief Psychotic Disorder, Shared Psychotic Disorder, and mania in bipolar disorder, the method comprising administering a therapeutically effective amount of the salt of claim 9.

65. A method of manufacturing the salt of claim 9, wherein the method comprises-preparing and isolating the salt.

66. The method of claim 65, further comprising crystallizing a base of Compound I by precipitating the base of Compound I from a solvent; and separating the solvent from the precipitated crystalline base of Compound I.

67. The method of claim 66, wherein the solvent comprises heptane.

68. The method of claim 65, further comprising making a pharmaceutical composition comprising the salt and a pharmaceutically acceptable adjuvant, diluent, carrier, additive, or combination thereof.

69. A method of manufacturing Compound I, the method comprising setting free a base of Compound I; precipitating the base of Compound I in crystalline form; optionally recrystallizing the crystalline base of Compound I; and transferring the crystalline base of Compound I into a salt of Compound I, wherein the salt is as defined in claim 9.

70. The method of claim 69, wherein the base of Compound I is set free from a crude salt or a crude mixture of Compound 1.

71. A method of manufacturing a compound of formula II: 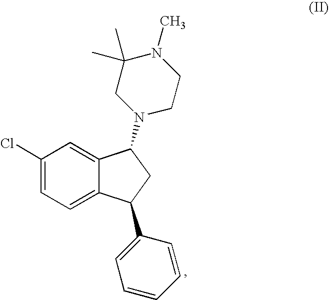
the method comprising methylating a secondary amine of Compound I: 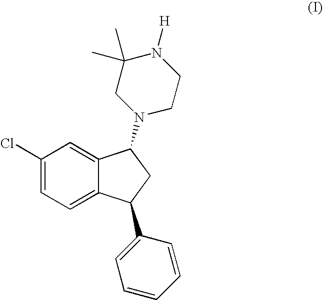
to obtain a free base of the compound of formula II, wherein Compound I is prepared by the method of claim 65.

72. The method of claim 71, further comprising precipitating the compound of formula II as a salt.

73. The method of claim 72, wherein the salt is a succinate or a malonate salt.

74. The method of claim 71, further comprising making a pharmaceutical composition comprising the compound of formula II or a salt thereof and a pharmaceutically acceptable adjuvant, diluent, carrier, additive, or combination thereof.
